(19) United States
(12) Patent Application Publication
(10) Pub. No.: US 2007/0005097 A1
(43) Pub. Date: Jan. 4, 2007

(54) Intravascular filter

(76) Inventor: Richard J. Renati, Los Gatos, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/156,426

(22) Filed: Jun. 20, 2005

(51) Int. Cl. A61M 29/00

(52) U.S. Cl. 606/200

(57) ABSTRACT

Intravascular filters such as embolic protection filters can be configured to capture embolic debris when deployed within a patient's vasculature. In particular, intravascular filters such as embolic protection filters can be configured to reduce or eliminate emboli becoming ensnared in the stagnant flow patterns that can otherwise arise near a filter-vascular wall junction.

### TECHNICAL FIELD

The invention relates generally to intravascular filters and methods of their formation. In particular, the invention relates to intravascular filters configured for improved vasculature interaction.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel.

Because of this debris, a number of devices, such as intravascular filters, have been developed to filter out this debris. A need remains for improved intravascular filters. A need remains for improved methods of manufacture of intravascular filters.

### SUMMARY

The invention is directed to intravascular filters such as embolic protection filters that are configured to capture embolic debris when deployed within a patient's vasculature.

Accordingly, an example embodiment of the invention can be found in a filter assembly that includes a support hoop, a filter membrane that has a proximal end that is secured to the support hoop, and stagnation prevention means that extend proximally from the support hoop.

Another example embodiment of the invention can be found in an embolic protection filter that includes a support hoop, a filter membrane having a proximal region secured to the support hoop, and a filter fillet that is positioned proximate the support hoop and that extends proximally from the support hoop.

Another example embodiment of the invention can be found in a method of forming a filter. A support hoop is provided. A filter membrane is formed, where the support hoop is positioned such that a proximal region of the filter membrane forms around the support hoop and encapsulates the support hoop. The filter membrane includes an extension that extends proximally from the support hoop.

Another example embodiment of the invention can be found in a method of capturing stagnant emboli. A filter is provided that includes a support hoop and a filter membrane. The filter membrane includes an extension portion that extends proximally from the support hoop. The filter is deployed within a vasculature including a vascular wall, where the proximal portion of the filter membrane and the extension portion contact the vascular walls. The extension portion is configured to prevent emboli from stagnating proximate the support hoop and the vascular wall.

### BRIEF DESCRIPTION OF THE FIGURES

The invention may be more completely understood in consideration of the following detailed description of various embodiments of the invention in connection with the accompanying drawings, in which:

FIG. 1 is a perspective view of an intravascular filter in accordance with an embodiment of the invention;

FIG. 2 is a closer view of a portion of the filter membrane included in the intravascular filter of FIG. 1;

FIG. 3 is a perspective view of the intravascular filter of FIG. 1, shown deployed within an artery or vein; and

FIG. 4 is an axial cross-section view of a portion of FIG. 3.

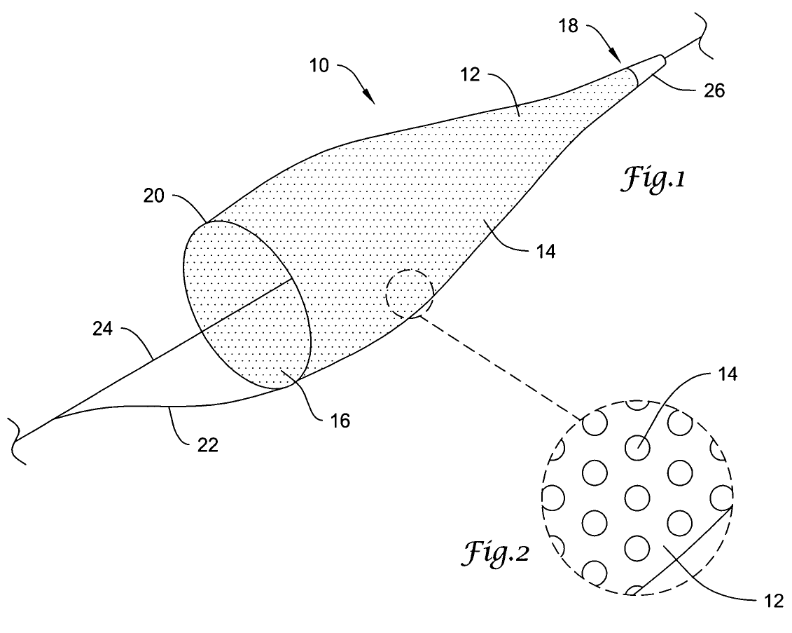

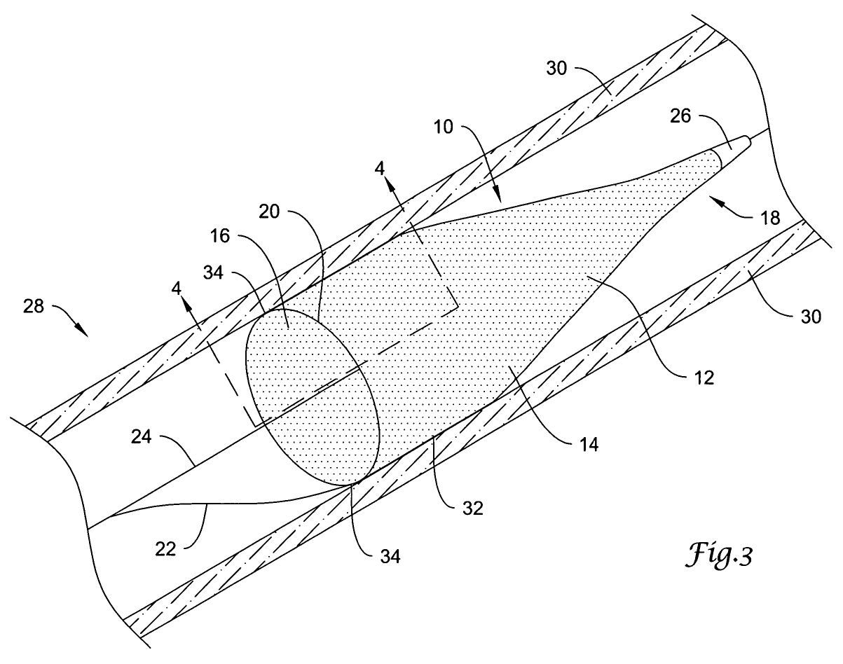

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

FIG. 1 is a perspective view of an example intravascular filter 10, which includes a filter membrane 12. The filter membrane 12 can be formed from any suitable moldable material or combination of materials. For example, the filter membrane 12 can include polymers such as polyether block amide, polybutylene terephthalate/polybutylene oxide copolymers sold under the Hytrel and Arnitel trademarks, Nylon 11, Nylon 12, polyurethane, polyethylene terephthalate, polyvinyl chloride, polyethylene naphthalene dicarboxylate, olefin/ionomer copolymers, polybutylene terephthalate, polyethylene naphthalate, ethylene terephthalate, butylene terephthalate, ethylene naphthalate copolymers, polyamide/polyether/polyester polyamides, aromatic polyamides, polyurethanes, aromatic polyisocyanates, polyetheretherketone, polycarbonates, polyamide/polyether, and polyester/polyether block copolymers, among others.

In some embodiments, the filter membrane 12 can be formed from at least one of polyether block amide, olefin/ionomer copolymers, nylon, polyurethane, polyethylene terephthalate, polyvinyl chloride, polyethylene naphthalene dicarboxylate and mixtures or copolymers thereof.

The filter membrane 12 can be porous, having pores 14 that are configured to permit blood flow while retaining embolic material of a desired size. The pores 14 are seen in greater detail in FIG. 1. While the pores 14 as illustrated are at least substantially circular in profile, other profiles are contemplated as well. The filter membrane 12 can have a mouth 16 and a closed end 18 and is capable of moving between an open state and a closed state. The mouth 16 can be sized to occlude the lumen of the body vessel in which the filter may be installed, thereby directing all fluid and any emboli through the filter.

A support hoop 20 can be attached to the filter membrane 12 at or proximal to the mouth 16. The support hoop 20 can be attached to the filter membrane 12 through melt bonding or other suitable means. In some embodiments, as discussed in greater detail hereinafter, the support hoop 20 can be integrally molded within the filter membrane 12. The support hoop 20 has an expanded state and a compressed state. The expanded state of the support hoop 20 is configured to urge the mouth 16 to its full size, while the compressed state permits insertion into a small lumen.

The support hoop 20 can be made from a flexible metal such as spring steel, from a super-elastic elastic material such as a suitable nickel-titanium alloy, or from other suitable material. The support hoop 20 can be a closed hoop made from a wire of uniform diameter, it can be a closed hoop made from a wire having a portion with a smaller diameter, it can be an open hoop having a gap, or it can have another suitable configuration.

A strut 22 can be fixedly or slideably attached to and extend from the support hoop 20. An elongate member 24 can be attached to and extend from the strut 22. The elongate member 24 can be attached to the strut 22 at an angle or the strut 22 can have a small bend, either at a point or over a region. The strut 22 can be attached to the support hoop 20 at a slight angle such that when the elongate member 24, the strut 22, and the support hoop 20 are in an unconstrained position, the elongate member 24 can generally extend perpendicular to the support hoop 20.

In the unconstrained position, the elongate member 24 can also lie along an axis which passes through the center of the region created by the support hoop 20. This may help position the support hoop 20 in contact with the wall of a vascular lumen or it may help in enhancing predictability or reliability during deployment. In some embodiments, the elongate member 24 can terminate at the strut 22. In other embodiments, the elongate member 24 can extend through the filter membrane 12, as shown. Whether or not the elongate member 24 extends through the filter membrane 12, it may be fixedly or slideably/rotatably attached to the filter membrane 12.

The filter membrane 12 can include a waist 26 at the closed end 18. In some embodiments, the waist 26 can be integrally formed with the filter membrane 12. In other embodiments, the filter membrane 12 can be further processed to form the waist 26. In some embodiments, integrally forming the waist 26 with the filter membrane 12 can reduce the outer diameter of the filter device when in a compressed state, increase the reliability and uniformity of the bond between the filter membrane and the elongate member, and reduce the number of steps or components needed to form the filter device.

The waist 26 is a region largely incapable of moving between two states and having a lumen of substantially constant diameter therethrough. The elongate member 24 can extend through and be bonded to the waist 26. This bonding can be heat bonding such as laser bonding or may be an adhesive or other suitable means.

With respect to FIG. 3, the intravascular filter 10 can be deployed within a vasculature 28 that can include an artery or a vein within a patient. For illustration purposes only, the vasculature 28 will be referred to herein as the vessel 28. In some embodiments, the vessel 28 can include vessel walls 30. As can be seen, the open mouth end 16 can be in substantial contact with the vessel walls 30 at a contact point 34. In some embodiments, at least a proximal portion 32 of the filter membrane 12 can also be in substantial contact with the vessel walls 30, depending on the particular geometry of the vessel 28.

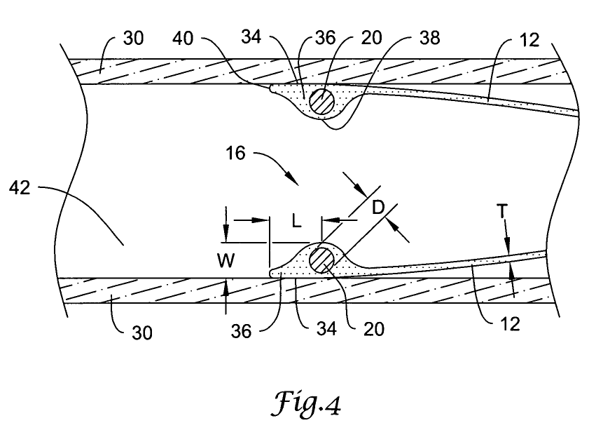

FIG. 4 is an axially aligned partial section view of FIG. 3, taken near the contact point 34. In this illustration, the support hoop 20 can be seen embedded within the filter membrane 12. In some embodiments, a fillet 36 that is integrally formed with the filter membrane 12 can extend proximally beyond the support hoop 20. The fillet 36 can extend circumferentially at least partially around the support hoop 20. In some embodiments, the fillet 36 can extend circumferentially substantially or completely around the support hoop 20.

As can be seen in FIG. 4, the fillet 36 provides a more gradual transition to the open mouth end 16 of the intravascular filter 10. The fillet 36 can taper from a more distal position 38 proximate the support hoop 20 to a proximal position 40. At the distal position 38, the fillet 36 can have a material thickness T that is about equal to that of the filter membrane 12. The fillet 36 can have an overall thickness W at the distal position 38 that is about equal to a cross-sectional diameter D of the support hoop 20 plus twice the thickness T of the filter membrane 12.

At the proximal position 40, the fillet 36 can have a thickness W that tapers to about zero. The fillet 36 can have any suitable length L. In some embodiments, the fillet 36 has a length L that is about one to four times a cross-sectional diameter D of the support hoop 20. In particular embodiments, the fillet 36 can have a length L that is about one to two times the cross-sectional diameter D of the support hoop 20.

The intravascular filter 10 can be built to any suitable dimensions. In some embodiments, the support hoop 20 can have a cross-sectional diameter D that is in the range of about 0.001″ to about 0.010″. In some embodiments, the filter membrane 12 can have a thickness T that is in the range of about 0.0004″ to about 0.003″. In such embodiments, the fillet 36 can have an overall thickness W that is in the range of about 0.002″ to about 0.016″ (equal to D+2T) and a length L that is in the range of about 0.001″ to about 0.004″ (1D to 4D) and in particular embodiments a length L that is in the range of about 0.010″ to about 0.040″ (1D to 2D).

The fillet 36 can be configured with any suitable shape or profile, provided that the shape or profile provides for relatively smooth blood flow between a region 42 that is upstream of the intravascular filter 10 and the open mouth end 16 of the intravascular filter 10. In some embodiments, providing for relatively smooth blood flow in this region can reduce or eliminate the formation or collection of emboli that could otherwise form or collect just upstream of the contact point 34. As shown, the fillet 36 tapers linearly from the distal position 38 to the proximal position 40. In other embodiments, the fillet 36 can taper in a concave or convex configuration between the distal position 38 and the proximal portion 40.

The filter membrane 12 can be formed in any suitable manner. In some embodiments, the filter membrane 12 can be formed using spray molding in which an appropriately shaped mandrel is provided. A number of layers of polymeric material are sprayed onto the mandrel to form the filter membrane. After several layers of polymeric material are sprayed onto the mandrel, the support loop 20 can be positioned over the mandrel, and additional polymeric layers can be sprayed onto the mandrel. In some embodiments, the fillet 36 can be formed by positioning the support loop 20 an appropriate distance distally of the proximal end of the sprayed polymeric layers. As a result, the support loop 20 is encapsulated within the filter membrane 12.

In some embodiments, the tapered profile of the fillet 36 can be created by masking during the spray molding process. In some embodiments, the tapered profile of the fillet 36 can be formed in a post-spraying grinding or milling process.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

We claim:

1. A filter assembly, comprising:
a support hoop;
a filter membrane having a distal end and a proximal end, the proximal end secured to the support hoop; and
stagnation prevention means extending proximally from the support hoop.

2. The filter assembly of claim 1, wherein the proximal end of the filter membrane envelops the support hoop.

3. The filter assembly of claim 2, wherein the stagnation prevention means extends proximally from the proximal end of the filter membrane.

4. The filter assembly of claim 1, wherein the stagnation prevention means comprises a material that is the same as that of the filter membrane.

5. The filter assembly of claim 4, wherein the stagnation prevention means comprises an extension of the filter membrane.

6. An embolic protection filter comprising:
a support hoop;
a filter membrane having a distal region and a proximal region, the proximal region of the filter membrane secured to the support hoop; and
a filter fillet positioned proximate the support hoop and extending proximally therefrom.

7. The embolic protection filter of claim 6, further comprising a guidewire.

8. The embolic protection filter of claim 7, wherein the distal region of the filter membrane is secured to the guidewire.

9. The embolic protection filter of claim 7, further comprising a strut extending from the guidewire to the support hoop.

10. The embolic protection filter of claim 6, wherein the proximal region of the filter membrane encompasses the support hoop.

11. The embolic protection filter of claim 6, wherein the filter fillet comprises an integral extension of the filter membrane.

12. The embolic protection filter of claim 6, wherein the filter fillet has a distal end and a proximal end, and the distal end of the filter fillet is positioned proximate the proximal region of the filter membrane.

13. The embolic protection filter of claim 12, wherein the filter fillet has a distal thickness at its distal end and a proximal thickness at its proximal end, and the proximal thickness is less than the distal thickness.

14. The embolic protection filter of claim 12, wherein the filter fillet tapers from a first thickness that is approximately equal to or greater than a diameter of the support loop to a second thickness that is approximately zero.

15. The embolic protection filter of claim 6, wherein the filter fillet has a length defined between a proximal end of the filter fillet and the support hoop, where the length is about one to four times a cross-sectional diameter of the support hoop.

16. The embolic protection filter of claim 15, wherein the filter fillet length is about one to two times the cross-sectional diameter of the support hoop.

17. A method of forming a filter, comprising steps of:
providing a support hoop; and
forming a filter membrane having a distal region and a proximal region, where the support hoop is positioned such that the proximal region of the filter membrane forms around the support hoop, thereby encapsulating the support hoop;
wherein the filter membrane includes an extension that extends proximally from the support hoop.

18. The method of claim 17, wherein forming a filter membrane comprises a spray molding process.

19. The method of claim 18, wherein the extension is shaped via masking during the spray molding process.

20. The method of claim 18, wherein the extension is shaped via a grinding or milling process subsequent to the spray molding process.

21. The method of claim 18, wherein the extension is shaped to reduce stagnant flow proximate the extension when the filter is deployed.

22. A method of capturing stagnant emboli, comprising steps of:
providing a filter comprising a support hoop and a filter membrane having a proximal portion, the filter membrane including an extension portion extending proximally from the support hoop;
deploying the filter within a vasculature including vascular walls, such that the proximal portion of the filter membrane and the extension portion contact the vascular walls; and
removing the filter;
wherein the extension portion is configured to reduce stagnant flow proximate the support loop and vascular wall.
